(19) United States
(12) Patent Application Publication
(10) Pub. No.: US 2002/0119488 A1
(43) Pub. Date: Aug. 29, 2002

(54) EX-CORPORE METHOD FOR TREATING HUMAN BLOOD CELLS

(76) Inventors: LOTHAR KOCH, WEINGARTEN (DE); WILLEM JANSSENS, FORST (DE); JACQUES VAN GEEL, ETTLINGEN-OBERWEIER (DE); FREDDY DEZEURE, TERVUREN (BE); DANNY VANSTRAELEN, HASSELT (BE)

Correspondence Address: SUGHRUE MION ZINN MACPEAK & SEAS, 2100 PENNSYLVANIA AVENUE NW, WASHINGTON, DC, 200373202

(21) Appl. No.: 09/297,557

(22) PCT Filed: Nov. 10, 1997
(86) PCT No.: PCT/EP97/06221

(30) Foreign Application Priority Data
Nov. 15, 1996 (EP) 96118377.9

(51) Int. Cl. G01N 33/53; A61K 39/00; C07K 16/00

(52) U.S. Cl. 435/7.1; 530/387.1

(57) ABSTRACT

This invention relates to an ex-corpore method of treating human blood cells incorporating tumour cells particularly in the frame of High-Dose Chemotherapy and/or Total Body Irradiation

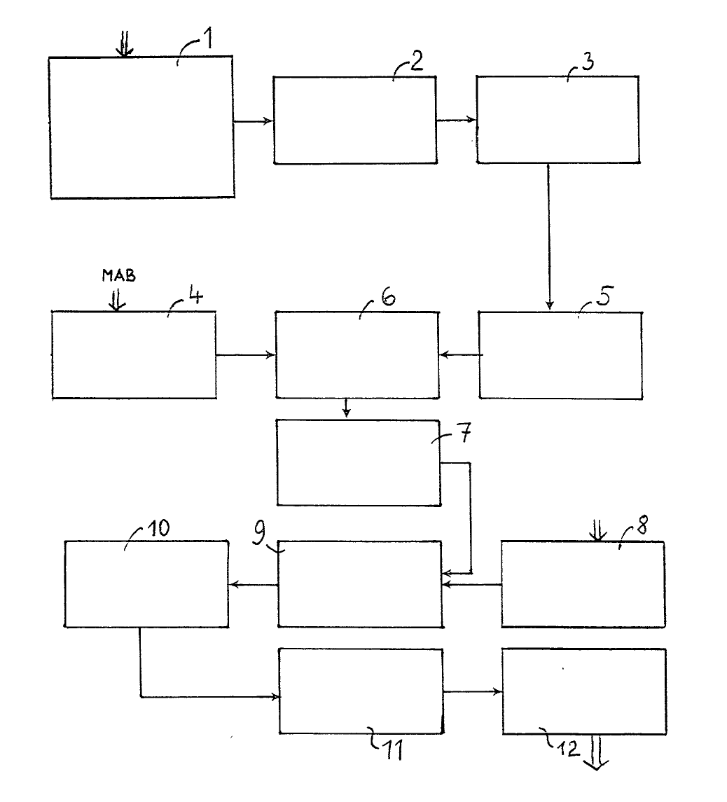

According to the invention,

a) carriers selective for specific tumour cells are conjugated to radionuclides which emit short range alpha particles with high linear energy transfer, forming a radioimmunoconjugate,

b) a filtration/separation step is performed on the radioimmunoconjugate ensuring the removal of non-conjugated radionuclides,

c) stem cells extracted from said blood and/or bone marrow, extracted from a patient for autologous transplant, are mixed ex-corpore with said conjugated radionuclides,

d) after an incubation period a purging of the blood cells and/or bone marrow is performed.

This invention relates to an ex-corpore method for purging human blood cells with respect to tumour cells using radioactive nuclides killing the tumour cells by use of radioactive nuclides.

Blood cells referred to in this text include both stem cells (e.g. peripheral stem cells harvests directly from the patient's blood, with or without the use of haematopoietic or colony-stimulating growth factors) and bone marrow (e.g. extracted from the patient for autologous transplant).

Purging of blood cells from tumour cells is of crucial importance for the success of high dose chemotherapy (HDCT) and total body irradiation (TBI) during autologous bone marrow transplantation (ABMT) and haematopoietic stem cell rescue (HSCR), see for example the papers:

“New strategies in marrow purging for breast cancer patients receiving high-dose chemotherapy with autologous bone marrow transplantation” of E. J. Shpall, S. M. Stemmer, S. I. Bearman, S. Myers, M. Purdy, R. B. Jones, published in Breast Cancer Research and Treatment, 1993, 26 suppl S.19-23d

“Autologous bone marrow transplantation vs continued chemotherapy for stage IV neuroblastoma: a nonrandomized childrens cancer group study” of D. O. Stram, K. K. Matthay, M. C. O'Leary, C. P. Reynolds, G. M. Haase, J. B. Atkinson, R. C. Seeger published in Proc Annu Meet Am Soc Clin Oncol 1995, page 14 (A1435)

“Purging of small cell lung cancer cells from human bone marrow using ethiofs (WR-2721) and light-activated merocyanine 540 phototreatment” of R. C. Meagher, S. A. Rothman, P. Paul, P. Koberna, C. Willmer, P. A. Baucco published in Cancer Research (Jul. 1, 1989 ) 49 (13) 3637-41.

However, the removal of tumour cells and residues during purging has not yet been successfully achieved until now, see for example the papers:

“Die Hochdosischemotherapie in der Behandlung des Mammakarzinoms: Gegenwärtiger Stand und Grenzen der Therapiemethode” of H. G. Derigs, C. Huber, M. Mahlke, K. Kolbe published in Geburtshilfe und Frauenheilkunde April 1996 56(4) 190-7

“High-dose chemotherapy with stem-cell rescue for the treatment of breast cancer” of D. L. McCauley published in Amarican Journal of Health-System Pharmacy Mar. 1, 1996 53(5) 521-34

“Autologous bone marrow transplantation in the treatment of acute myeloid leukaemia: the Dartmouth experience and a review of literature” of L. E. Mills, G. G. Cornwell published in Cancer Investigation (1990) 8 (2) 181-90.

²¹²Bi and ²¹³Bi conjugated to monoclonal antibodies have proven to be effective in killing cancer cells in vitro and are about to be applied as in-vivo therapy (see “Cytotoxicity of ²¹³Bi and ²²⁵Ac immunoconjugates” of F. M. Kaspersen, E. Bos, A. V. Doornmalen, M. W. Geerlings, C. Apostolidis, R. Molinet published in Nuclear Medicine Communications 16, 1995). A similar method is proposed in EP 0 585 986 A or in FR-A-2 527 928. This would involve the injection of radioactivity in a patient where it could lead to damage of healthy cells located in organs which have some absorption properties for the monoclonal antibodies and their conjugates.

The invention as defined in the annexed claims proposes a new method of purging blood which excludes such side-effects. Compared to the above quoted methods the main idea of the invention is to treat ex-corpore, with the help of a cancer cell killing radionuclide, an autologous transplant (blood stem cells and/or bone marrow) which is finally re-injected into the patient practically free of artificial radioactivity.

This treatment is fundamentally different from the well-known “extracorporel immunoadsorption” (ECIA) described e.g. in Database Medline US NML AN=96279681 XP002030431 ans Journal of Nuclear Medicine, Vol.34 No 3, pages 448 to 454. In fact, ECIA is a procedure of plasma replacement/cleaning done after having injected radioactivity and monoclonal antibodies into the patient's body (in vivo), to reduce the non-tumor specific uptake in the patient (by taking away radioisotopes ex-corpore) and therefore improve the tumor to background ratio in the patient.

This invention can be applied in Autologous Bone Marrow Transplantation (ABMT) and/or Haematopoietic Cell Rescue (HSCR) performed in the frame of High-Dose Chemotherapy (HDCT) and/or Total Body Irradiation (TBI). In the described application, killing the tumour cells contaminating the bone marrow and the stem cells takes place ex-corpore. The tumour cells are destroyed by short-lived radionuclides, which spare largely other cells due to the short decay range and the selectivity of the carriers for the tumour cells.

During HDCT and/or TBI the blood cells for the autologous transplant are cryopreserved for several days. During this period they can be purged at external facilities equipped to handle such radioactive nuclides, eliminating the need of hospitals to install and maintain expensive specialised laboratories. This is contrary to the in-vivo treatment, which requires an on-site preparation of the radioimmunoconjugate loaded with the alpha-emitter (e.g. ²¹³Bi) prior to injection because of the short half life and the instability of the conjugate.

The required activity for the purging method, as described in this invention, is expected to be considerably smaller than in the case of the in-vivo treatment. The main reason is that only the bone marrow and/or stem cells are treated to kill the cancer cells. The major amount of cancer cells in the patient will indeed be killed in the associated HDCT and/or TBI.

If the half lives of the radionuclides used for the purging are short enough, the transplant will be practically free of artificial radioactivity and does not expose the patient in excess of the maximum permissible annual limit for intake of these nuclides (e.g. Richtlinie 96/29/Euratom des Rates vom 13. Mai 1996, referred to in Amtsblatt der Europäischen Gemeinschaften, L159, Jun. 29, 1996).

The possible long term effects of the residues of the alpha-emitters in the patient's body (mainly heavy metals) are not yet known. It is clear however than any associated risk will be strongly reduced when the alpha-activity is only used for the purging (as described in this invention) compared to the in-vivo treatment.

A dose of less than 100 mCi of ²¹²Bi/²¹³Bi is estimated to be sufficient for purging. This activity will decay to the maximum permissible annual uptake of alpha-emitting B-nuclides of 10⁻⁶Ci within one day regardless of any purification of the transplant after purging. This avoids the patient to be exposed beyond limits for artificial radioactivity.

A central laboratory is provided to use the products and implement the techniques described here. The structure of this central laboratory depends on the radionuclides used. Two examples are given here. When using pure ²¹³Bi (e.g. separated from irradiated ²²⁶Ra targets or ²³³U decay), the central laboratory is schematically presented in the enclosed unique figure of the drawings. It consists of three installations, grouped under one roof. If a mixture of ²¹²Bi and ²¹³Bi were used (e.g. separated after neutron irradiation of a ²²⁶Ra target—see patent EP 0 443 479 B1) the set-up of the central laboratory would be slightly different. The main differences are the source materials for the radionuclides and the use of shielded facilities in the case of the ²¹²Bi/²¹³Bi mixture. The operations to be performed in the sterile installations are similar in both cases.

The installation shown in the drawings includes in a first part all the equipment, tools and materials to produce the required radionuclides upon request:

a hot cell 1 to separate the ²¹³Bi parent nuclides from the source material,

a glovebox 2 to fabricate the ²¹³Bi generator,

a set of analysis tools 3 (e.g. α and γ spectrometers) to determine the radiochemical yield and purity of the source materials and daughter products.

In the case where ²²⁹Th is used as mother nuclide, the following actions are performed:

²²⁹Th is absorbed from 8 M HNO₃ on a Dowex 1x8, 100-200 mesh resin batch (for 63 mg Th, about 1 ml resin is taken). The breakthrough of about 2 percent Th is removed by passing through a second 1.5×20 cm column of the same resin. The effluent contains ²²⁵Ra, ²²⁵Ac and decay-products (i.e. most ²²⁵Ac). Eventual Th traces still present in the effluent are absorbed on a third column of Dowex 50 Wx8, 100-200 mesh resin column (0.5×30 cm).

The chemical purity of the reagents for this production is known and checked by Inductively Coupled Plasma Mass Spectrometry.

The radiochemical purity, already at this stage, is determined using alpha- and gamma-spectrometry.

According to a variant, a second source of ²²⁵Ac is the direct production by irradiation of ²²⁶Ra in a cyclotron. A chemical separation step in this case is required to separate the Ra from the Ac. This is done in the following way:

Boiling the Ac-Ra-mixture in 8 M HNO₃,

Evaporating the Ac³⁺ and Ra²⁺ in the solution and redissolving in 2 M HCl

Fixing this product onto a previously conditioned column filled with Dowex 50Wx8 (100-200 mesh SERVA)

Passage of 1 bed volume 2 M HCl eliminates ²²¹Fr, ^{212,213}Bi, ²⁰⁹Pb

Passage of 10 bed volumes 2 M HNO₃ eliminates ²²⁶Ra

Elution of the ²²⁵Ac product is then performed by passing 5 bed volumes 4 M HNO₃.

²¹³Bi can be obtained from ²²⁵Ac according to the following method:

The Ac, which is fixed typically on a cation exchanger, can be “milked” to extract ²¹³Bi with both a good yield and high purity.

A glass column (50*5 mm) with 0.4 g Dowex 50 Wx8 (100-200 mesh SERVA) is used which is conditioned by successive passages of 5 bed volumes 9 M HCl, 5×H₂O and 5×2 M HCl.

The ²²⁵Ac product is evaporated, redissolved in 0.2 ml HCl and fixed on to the column.

The accumulation of purified ²²⁵Ac is followed by a quality control procedure consisting of alpha-spectrometry, gamma-spectrometry and/or Inductively Coupled Plasma Mass Spectrometry.

Thereafter the produced short lived alpha-emitting radionuclides must be coupled to the monoclonal antibodies (MAB) or any other appropriate carriers. The MAB and other carriers are conjugated to the appropriately treated and conditioned radionuclides. The radioimmunoconjugate undergoes extensive quality control before being transferred to the next installation. This part of the laboratory therefore possesses:

a sterile bank 4 to prepare the MAB or other carriers after receipt from outside

a glovebox 5 to produce the short lived radionuclides (here ²¹³Bi)

a glovebox 6 to conjugate the ²¹³Bi to the MAB or other carriers

A quality control station 7 to check e.g. the stability and loading efficiency of the radioimmunoconjugate.

The following actions are performed:

²¹³Bi is eluted from the above described generator in 2 bed volumes 0.1 M NaI/HCl.

This elution procedure is repeated until the ²²⁵Ac activity is exhausted.

To the monoclonal antibodies or other carriers chemical ligands are conjugated (e.g. chelates) in order to load the radionuclide. Alternatively, the chemically complexed radionuclide is condensed to —SH, —NH oder —OH groups of the monoclonal antibodies or other carriers.

The conjugate has to be purified from not reacted material and sterilised.

Monoclonal antibodies and other carriers for different cancer types are commercially available and some have been used for therapeutic purposes. They will be selected according to the cancer type for the subsequent purging process taking into account:

High selectivity of the antibodies to the tumour cell type

Stability of the conjugate complex in retaining ²¹³Bi

The method is described for conjugation of BB4 IgG with Benzyl DPTA complex and ²¹³Bi.

²¹³Bi is brought in solution in a colloid-free form and adjusted to pH 5 by the addition of 50 microliter of 10 M NaOH and 200 microliter of a citrate/acetate buffer 0.25/1.3 M, pH 5.

The immunoconjugate is brought to the appropriate conditions to be mixed with the radioisotope solution described above (a different approach can be followed in first complexing the radioisotope with an organic compound which is condensed to the unchanged monoclonal antibody or other carrier).

This mixture of the radioisotope solution and the immunoconjugate can be incubated

The quality of the loading of ²¹³Bi to the immunoconjugate is being checked by TLC.

To remove the non-bound ²¹³Bi of bound ²¹³Bi to the radioimmunoconjugate, a filtration/separation step is provided, using a working principle similar to that of a molecular sieve such as Sephadex G-100 or PD-10 exclusion-diffusion gel, or similar.

Analysis will check for radiochemical impurities by gamma-spectrometry and alpha-spectrometry.

Thereafter, the actual purging of the blood cells is performed. After stem cell harvest or bone marrow extraction the blood cells arrive from the hospitals and will be prepared for the mixing with the conjugated nuclide. After this mixing, the purging of the blood stem cells/bone marrow takes place, the crucial aspect being that this treatment takes place outside of the patient's body. The treated transplant will then be subject to a quality control before being prepared to be shipped back to the hospital (in cryo-preserved form). For this purpose the following equipment, techniques and materials are required in this installation:

a receipt equipment 8 for the blood cells

a mixing equipment 9 for the blood cells and the conjugated radionuclide

a purging equipment 10 (e.g. incubators—see below) for the transplant after the treatment

a quality control station 11 to check the purification of transplant

an equipment 12 for cryo-preservation and for preparing the transplant for transport before sending it back to the hospital.

The following actions are performed:

Prior to the purging of the blood cells (bone marrow and stem cells) the cytotoxicity of the ²¹³Bi immunoconjugate for the tumour cells is tested as follows:

For this purpose monolayers of the tumour cells are brought in contact with the radioimmunoconjugate.

The survival rate of the cells, brought in contact with the radioimmunoconjugate, is tested:

The MTT check, based on chemical interaction with the cells inducing colour changes, uses an absorption spectrometer to determine the survival rate.

The number of radionuclide atoms needed depends on the tumour size. Accordingly the selectivity of the immunoconjugate to the tumour cell in question has to be determined in advance.

From the foregoing description and the annexed claims it will be clear that the present invention differs from a medical treatment of human patients by the fact that the method is applied ex-corpore in a radiological laboratory on extracted blood stem cells and is not practised on the human body itself. This particularity offers decisive advantages with respect to the in-vivo treatment:

The amount of activity required (only to kill residual tumour cells in the bone marrow or stem cell transplant) is minimised and the risk to damage other tissues is largely eliminated contrary to in-vivo treatment.

The purged blood cells are returned to the patient body without radioactivity (i.e. below the maximum permissible annual limit for intake, see e.g. “Richtlinie 96/29/Euratom des Rates vom 13. Mai 1996” referred to in Amtsblatt der Europaischen Gemeinschaften, L159, Jun. 29, 1996) due to the use of short-lived radionuclides and the treatment ex-corpore.

The treatment is performed outside the hospital in a central laboratory having the required operating licenses and installation “under one roof” and being able to serve more than one hospital.

1. An ex-corpore method of treating human blood cells incorporating tumour cells, in particular in the frame of High Dose Chemotherapy and/or Total Body Irradiation, characterized by the following steps:
a) carriers selective for specific tumour cells are conjugated to radionuclides which emit short range alpha particles with high linear energy transfer, forming a radioimmunoconjugate,
b) a filtration/separation step is performed on the radioimmunoconjugate ensuring the removal of non-conjugated radionuclides,
c) stem cells extracted from said blood and/or bone marrow, extracted from a patient for autologous transplant, are mixed ex-corpore with said conjugated radionuclides,
d) after an incubation period a purging of the blood cells and/or bone marrow is performed.

2. A method according to claim 1, characterized in that the radionuclides are selected from ²²⁵Ac, ²¹²Bi, ²¹³Bi and mixtures thereof.

3. A method according to any one of the preceding claims, characterized in that chemical ligands, e.g. chelates, are conjugated to the carriers in order to load the radionuclides.

4. A method according to any one of claims 1 to 2, characterized in that the chemically complexed radionuclide is condensed to —SH, —NH or —OH groups of the carrier.

5. A method according to any one of claims 1 to 4, characterized in that the carriers are selected from monoclonal antibodies, hormones and proteins specific to the respective tumour cells.

6. A method according to any one of claims 1 to 5, characterized in that the conjugated radionuclides are sterilised prior to step c).
